(19) United States
(12) Patent Application Publication
(10) Pub. No.: US 2007/0231826 A1
(43) Pub. Date: Oct. 4, 2007

(54) Article and assembly for magnetically directed self assembly

(75) Inventors: William Hullinger Huber, Scotia, NY (US); Ching-Yeu Wei, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/254,094

(22) Filed: Oct. 19, 2005

(51) Int. Cl. G01N 33/53; C12M 3/00

(52) U.S. Cl. 435/7.1; 435/287.2

(57) ABSTRACT

An article for assembly includes a substrate, at least one receptor site disposed on the substrate and a patterned magnetic film. The patterned magnetic film includes at least one magnetic region, each magnetic region being disposed within one of the receptor sites. The patterned magnetic film comprises a material with a perpendicular magnetic anisotropy. An assembly includes at least one functional block comprising at least one element and a patterned magnetic film comprising at least one region. The assembly further includes an article comprising a substrate, at least one receptor site disposed on the substrate and at least one receptor configured to generate a magnetic field gradient for attracting the region. The receptor is positioned at the receptor site.

### STATEMENT REGARDING FEDERALLY SPONSORED RESEARCH & DEVELOPMENT

This invention was made with Government support under contract number W911CX04C0099 awarded by the Defense Advanced Research Agency for the Department of Defense. The Government has certain rights in the invention.

### CROSS REFERENCE TO RELATED APPLICATIONS

This application is related to commonly assigned, concurrently filed U.S. Patent Application to W. H. Huber et al., entitled “Functional blocks for assembly and method of manufacture,” which application is incorporated by reference herein in its entirety. This application is also related to commonly assigned, concurrently filed U.S. Patent Application to W. H. Huber et al., entitled “Methods for magnetically directed self assembly,” which application is also incorporated by reference herein in its entirety.

### BACKGROUND

The invention relates generally to the assembly of components onto a surface, and more particularly, to the assembly of building blocks onto a substrate for electronic circuit fabrication, sensors, energy conversion, photonics and other applications.

There is a concerted effort to develop large area, high performance electronics for applications such as medical imaging, nondestructive testing, industrial inspection, security, displays, lighting and photovoltaics, among others. Two approaches are typically employed. For systems involving large numbers of active elements (for example, transistors) clustered at a relatively small number of locations, a “pick and place” technique is typically employed, for which the active elements are fabricated, for example using single crystal semiconductor wafers, and singulated (separated) into relatively large components (for example, on the order of 5 mm) comprising multiple active elements. The components are sequentially placed on a printed circuit board (PCB). Typically, the components are sequentially positioned on the PCB using robotics. Because the pick and place approach can leverage high performance active elements, it is suitable for fabricating high performance electronics.

A key limitation of the pick and place approach is that the components must be serially placed on the PCB. Therefore, as the number of components to be assembled increases, the manufacturing cost increases to the point where costs become prohibitive. In addition, as the component size decreases, it becomes increasingly difficult to manipulate and position the components using robotics. Accordingly, this technique is ill-suited for the manufacture of low density, distributed electronics, such as flat panel displays or digital x-ray detectors. Instead, a wide-area, thin film transistor (TFT) based approach is typically employed to manufacture low-density, distributed electronics. Typically, the TFTs comprise amorphous silicon (a-Si) TFTs fabricated on large glass substrates. Although a-Si TFTs have been successfully fabricated over large areas (e.g. liquid crystal displays), the transistor performance is relatively low and therefore limited to simple switches. In addition, with this process, the unit cost of a large area electronic circuit necessarily scales with the size of the circuit.

Another approach is to substitute a higher mobility semiconducting material, such as polysilicon, cadmium selenide (CdSe), cadmium sulfide (CdS) or germanium (Ge), for a-Si to form higher mobility TFTs. While TFTs formed using these higher mobility materials have been shown to be useful for small-scale circuits, their transistor characteristics are inferior to single crystal transitors, and thus circuits made from these materials are inherently inferior to their single crystal counterparts. As with a-Si, the unit cost of a large area electronic circuit necessarily scales with the size of the circuit, for this process.

A number of approaches have been developed to overcome these problems. For example, U.S. Pat. No. 5,783,856, to Smith et al., entitled “Method for fabricating self-assembling microstructures,” employs a fluidic self-assembly process to assemble trapezoidal shaped components dispersed in a solution onto a substrate having corresponding trapezoidal indentations. This approach uses gravity and convective fluid flow to deposit the components in the indentions. Limitations of this technique include: the use of relatively weak forces to dispose and hold the blocks in the indentations. It would further appear to be difficult to assemble a large variety of elements to the substrate due to the limited number of block and indent shapes that can realistically be fabricated.

U.S. Pat. No. 6,657,289, to Craig et al., entitled “Apparatus relating to block configurations and fluidic self assembly process,” employs a fluidic self-assembly process to assemble components having at least one asymmetric feature dispersed in a solution onto a substrate having correspondingly shaped receptor sites. Limitations of this technique include: the use of relatively weak forces to dispose and hold the blocks in the shaped sites. It would further appear to be difficult to assemble a large variety of elements to the substrate due to the finite number of component shapes available.

U.S. Pat. No. 6,780,696, to Schatz, entitled “Method and apparatus for self-assembly of functional blocks on a substrate facilitated by electrode pairs,” employs another fluidic self-assembly process to assemble trapezoidal shaped components dispersed in a solution onto a substrate having corresponding trapezoidal indentations. However, this approach couples electrodes to the substrate to form an electric field. The approach further forms the components of high-dielectric constant materials, such that the components are attracted to higher electric field regions and are thus guided to the trapezoidal indents. In another embodiment, the component is formed of a low magnetic permeability material, and a high magnetic permeability layer is coupled to the bottom surface of the component. A static magnetic field is generated at a receptor site by covering the receptor site with a permanent magnet having a north and a south pole aligned such that the static magnetic field is aligned parallel to the surface of the receptor site. In another embodiment, a magnetic field is applied parallel to the substrate. The slurry solution has an intermediate value of magnetic permeability. A drawback of this technique is that the components will tend to agglomerate in solution, due to the propensity of high magnetic permeability materials to agglomerate so as to minimize magnetic energy. Another possible limitation on this technique is registration error between the component and the substrate resulting from the use of magnetic fields aligned parallel to the substrate. In addition this technique would not lend itself to the assembly of multiple component types.

U.S. Pat. No. 3,439,416, to Yando, entitled “Method and apparatus for fabricating an array of discrete elements,” forms pairs of magnets in a laminated base. Magnetic coatings, such as iron, are applied to the surface of elements. A multiplicity of elements is placed on the surface of the laminated base, which is then vibrated to move the elements. The magnetic coated surfaces of the elements are attracted to the pole faces of the magnet pairs. This technique suffers from several drawbacks, including severe limitations on the shape, size and distribution of the elements. For example, element width must match the spacing of the magnetic layers in the laminated base and the distribution of the elements is restricted by the parallel lamination geometry. In addition the technique appears to be applicable to relatively large, millimeter sized dimensions, and may not be suitable for smaller, micron-sized elements. In addition this technique would not lend itself to the assembly of multiple component types.

“Programmable assembly of heterogeneous colloidal particle arrays,” Yellen et al., Adv. Mater. 2004, 16, No. 2, January 16, p. 111-115, employs magnetically programmable assembly to form heterogeneous colloidal particle arrays. This approach utilizes micromagnets that are covered with an array of square microwells and which are magnetized parallel to the plane. The substrate is immersed in a bath, and superparamagnetic colloidal beads are injected into the bath. External magnetic fields are applied perpendicular to the plane in a first direction, causing the beads to be attracted to one pole of the micromagnets. The direction of the external magnetic field is then reversed, causing the beads to be attracted to the other pole of the micomagnets. A drawback of this technique is that it is limited to two types of particles. Another limitation of this technique is that it requires the application of external magnetic fields and appears to be limited to superparamagnetic colloidal beads. Another limitation on this technique is use of microwells to trap the beads. Yield would also appear to be an issue.

It would therefore be desirable to provide systems and methods for fabricating high performance, large area electronics rapidly and inexpensively. It would further be desirable for the improved systems and methods to facilitate the assembly of a variety of different types of elements.

### BRIEF DESCRIPTION

Briefly, one aspect of the present invention resides in an article for assembly. The article includes a substrate, at least one receptor site disposed on the substrate and a patterned magnetic film comprising at least one magnetic region. Each magnetic region is disposed within one of the receptor sites. The patterned magnetic film comprises a material with a perpendicular magnetic anisotropy.

Another aspect of the present invention resides in an assembly. The assembly includes at least one functional block comprising at least one element and a patterned magnetic film comprising at least one region. The assembly further includes an article comprising a substrate, at least one receptor site disposed on the substrate and at least one receptor configured to generate a magnetic field gradient for attracting the region. The receptor is positioned at the receptor site.

### DRAWINGS

These and other features, aspects, and advantages of the present invention will become better understood when the following detailed description is read with reference to the accompanying drawings in which like characters represent like parts throughout the drawings, wherein:

FIG. 1 schematically depicts an exemplary article embodiment of the invention with two exemplary receptor sites for attachment to two exemplary functional blocks;

FIG. 2 shows an exemplary assembly embodiment;

FIG. 3 depicts an exemplary article embodiment with one region disposed at each of the receptor sites;

FIG. 4 shows a plot of the magnetic field gradient as a function of distance above the center of an exemplary magnetic region;

FIG. 5 depicts an exemplary asymmetric quadrupole configuration with two magnetic regions arranged side-by-side at each of the receptor sites;

FIG. 6 depicts an exemplary symmetric quadrupole configuration with inner and outer magnetic regions;

FIG. 7 is a top view of an interconnect layer for an article;

FIG. 8 is a side view of the article;

FIG. 9 is a side view of an article embodiment of the invention with electromagnet-based receptors;

FIG. 10 schematically illustrates an example multilayer magnetic film;

FIG. 11 illustrates an exemplary functional block configuration;

FIG. 12 illustrates an exemplary dipole binding configuration for the magnetic moments of an exemplary functional block and article in side view;

FIG. 13 depicts an exemplary quadrupole configuration for the magnetic moment of an exemplary functional block;

FIG. 14 depicts another exemplary quadrupole configuration for the magnetic moment of an exemplary functional block;

FIG. 15 shows transistor electrodes formed on a functional block with symmetric contacts and a symmetric patterned magnetic region in a dipole configuration;

FIG. 16 illustrates one embodiment of an article configured for heterogeneous self-assembly;

FIG. 17 illustrates another embodiment of an article configured for heterogeneous self-assembly;

FIG. 18 depicts an exemplary diode array embodiment, configurable, for example, as a display or digital x-ray detector;

FIG. 19 shows transistor electrodes formed on a functional block with asymmetric contacts and a symmetric patterned magnetic region in a dipole configuration;

FIG. 20 shows an example assembly configuration with side contacts; and

FIG. 21 shows an example assembly configuration with back contacts.

### DETAILED DESCRIPTION

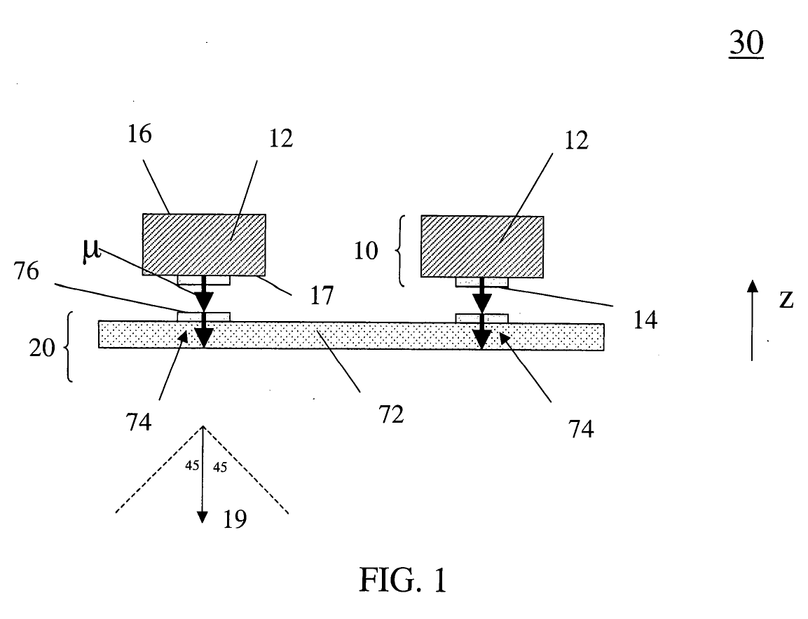

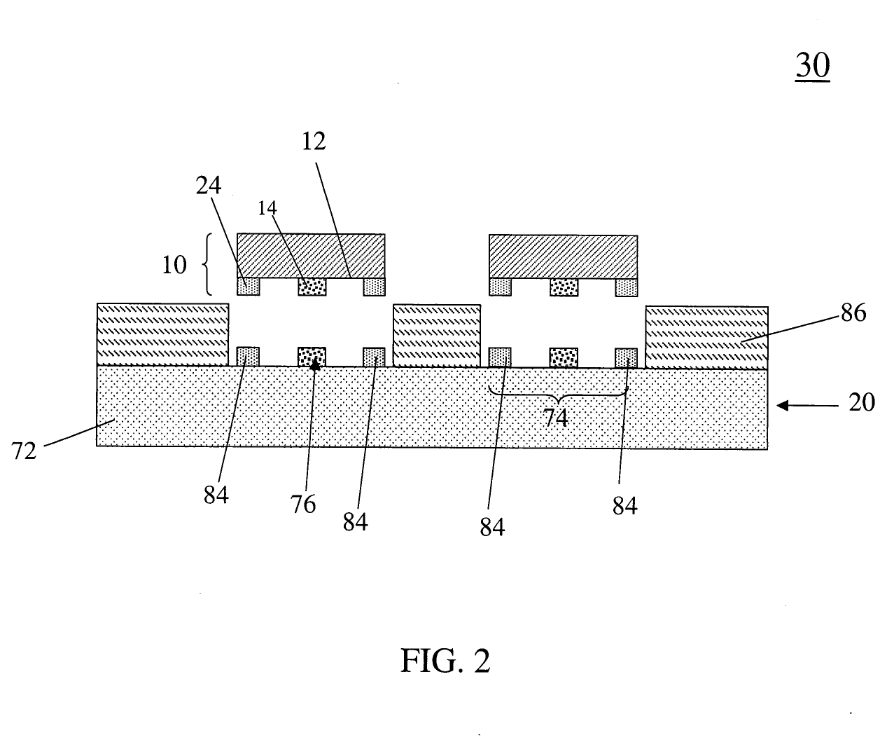

An article 10 embodiment of the invention is described with reference to FIGS. 1-8 and 16. The article 20 is configured for the magnetically directed self assembly (MDSA) of a number of functional blocks 10, as illustrated, for example in FIGS. 1 and 2. The MDSA method is described in commonly assigned, copending, concurrently filed U.S. Patent Application to W. H. Huber et al. entitled, “Methods for magnetically directed self assembly,” and the functional blocks 10 are described in commonly assigned, copending, concurrently filed U.S. Patent Application to W. H. Huber et al. entitled, “Functional blocks for assembly and method of manufacture.”

As shown, for example in FIG. 1, article 20 includes a substrate 72, at least one receptor site 74 disposed on the substrate 72 and a patterned magnetic film 76 comprising at least one magnetic region (also indicated by reference numeral 76) disposed within the receptor site. The patterned magnetic film 76 comprises a material with a perpendicular magnetic anisotropy. By “perpendicular magnetic anisotropy,” it is meant that it is energetically favorable to have the magnetic moment aligned perpendicular to the plane of the patterned film. Examples of materials with perpendicular magnetic anisotropy include, without limitation, Cobalt-Platinum multilayers, Cobalt-Palladium multilayers, Iron-Gadolinium-Terbium alloys and related rare-earth alloys.

Although FIG. 1 shows a patterned magnetic film 76 deposited on the substrate 72, the patterned magnetic film 76 may also be deposited on or otherwise affixed to an intermediate layer (not shown), such as a moisture and oxygen barrier layer, formed on the substrate 72. The patterned magnetic film 76 may be deposited on or other affixed to contacts formed on the substrate 72. Moreover, the term “deposited” also encompasses patterned magnetic films 76 that are partially or fully embedded in the substrate 72 (not shown).

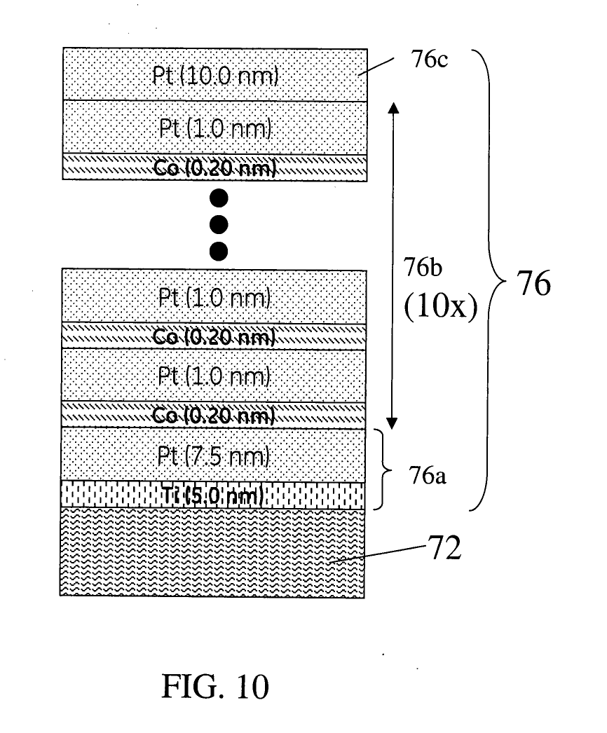

As used herein, the term “film” refers to a structure having one or more layers. FIG. 10 illustrates an exemplary multilayer film embodiment, for example. For particular embodiments, the thickness of the structure is less than or equal to 100 microns. In other embodiments, non-magnetic spacer layers (not shown) are included, and the thickness of the structure is less than about one mm. For the arrangement shown in FIG. 1, the thickness of the film 76 is measured in the z direction.

By “patterned” it is meant that the film has a shape such that it does not extend across the entire surface of the substrate 72. The film 76 can be patterned using lithographic techniques, for example. More particularly, the film 76 can be patterned by first forming an un-patterned film across at least a portion of the surface of the substrate 76 and then performing lithography.

### EXAMPLE

FIG. 10 shows an exemplary un-patterned multilayer magnetic film (also indicated by reference numeral 76 in FIG. 10) for forming patterned magnetic film 76. For the non-limiting, illustrated example of FIG. 10, a multilayer magnetic film 76 is constructed by sequentially depositing a series of individual layers comprising a base region 76a, an active region 76b and a capping region 76c. The multilayer film 76 may be deposited using a variety of different deposition processes, non-limiting examples of which include electron beam evaporation, sputtering, resistive source evaporation and electroplating.

In one non-limiting example, a perpendicular magnetic film may be generated via electron beam evaporation of Cobalt/Platinum multilayers in the active region 76b. For the example illustrated by FIG. 10, the base region 76a comprises approximately 5.0 nm of Titanium (Ti) followed by approximately 7.5 nm of Platinum (Pt). The active region 76b for this example comprises a multilayer stack wherein approximately 0.2 nm of Cobalt (Co) is alternated with approximately 1.0 nm of Pt to build an active region 76b with approximately 10 layers of Co. For this example, the capping region 76c comprises approximately 10 nm of Pt. For the exemplary structure of FIG. 10, the multilayer film 76 is formed on substrate 72.

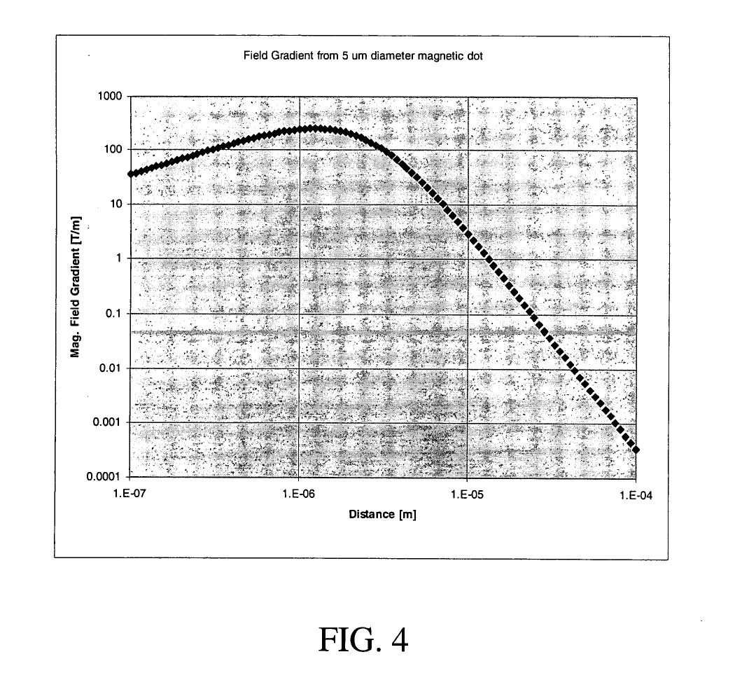

Returning now to the general description of the invention, according to particular-embodiments, each magnetic region 76 is configured to generate a magnetic field gradient. By “configured” it is meant that the specified magnetic field gradient for the patterned region 76 can be generated by application of an applied magnetic field and after such generation remains even in the absence of an applied magnetic field. According to a more particular embodiment, the magnetic field gradient for the patterned film 76 is configured to attract a patterned magnetic film 14 for a block 10 having a magnetic moment that is oriented in a range of plus or minus forty-five degrees from a surface normal 19 for the patterned magnetic film 14 for the block 10. For example, if the patterned magnetic region has a magnetization of approximately 1.44×10⁶ A/m, a diameter of approximately 5×10⁻⁶ m and a magnetic film thickness of approximately 2×10⁻⁹ m, the magnetic field gradient at a distance approximately 1×10⁻⁵ meter above the center of the region is approximately 2.9 Tesla/meter. A plot of the magnetic field gradient as a function of distance above the center of the magnetic region with the above parameters is shown in FIG. 4.

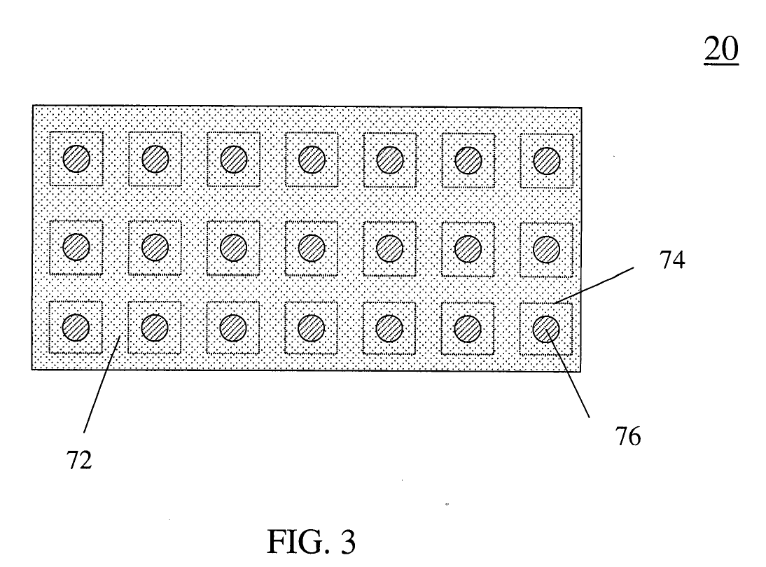

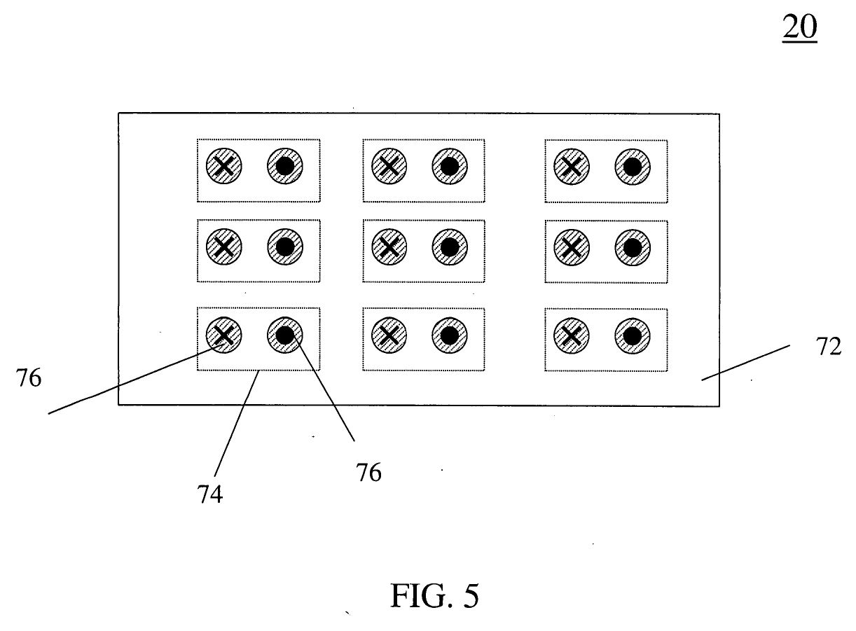

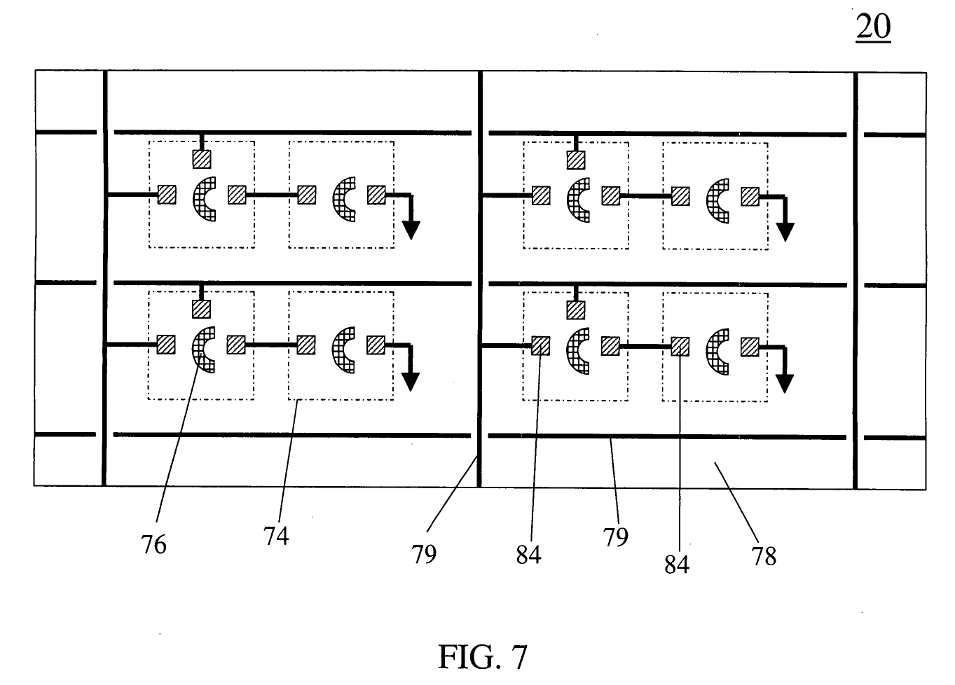

Different embodiments of article 20 have different numbers of magnetic regions 76 at the various receptor sites 74. For certain embodiments, the article 20 includes a number of receptor sites 74, and the patterned magnetic film 76 includes a number of magnetic regions 76. For the exemplary embodiment depicted in FIG. 3, for example, one symmetric magnetic region 76 is disposed at each of the receptor sites 74. In other embodiments, one asymmetric region (for example, as show in FIG. 7) is disposed at each receptor site. For other embodiments (see for example, FIG. 5), multiple regions are disposed at each receptor site. For these embodiments, the regions may be symmetric or asymmetric and arranged symmetrically or asymmetrically.

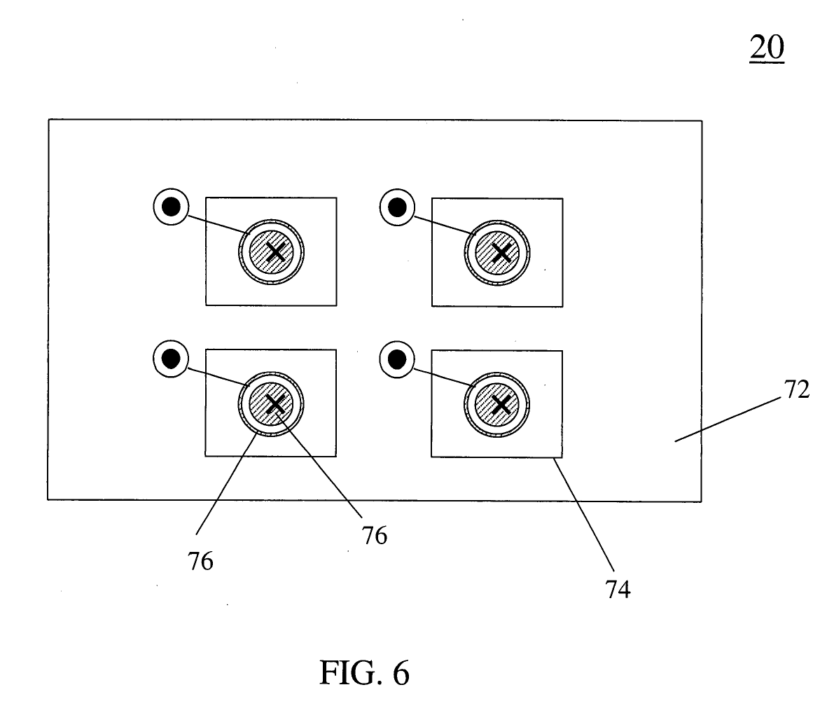

For other embodiments, a number of magnetic regions 76 are arranged in a quadrupole configuration for at least one of the receptor sites 74. For example, FIG. 5 depicts an exemplary quadrupole configuration with two side-by-side magnetic regions 76 at each of the receptor sites 74, where the two magnetic regions 76 have the same area (and hence volume) but have opposite directions of magnetization, as indicated. Similarly, FIG. 6 depicts another exemplary quadrupole configuration with two magnetic regions 76 at each of the receptor sites 74, where the inner and outer magnetic regions 76 have the same area (and hence volume) with opposite directions of magnetization. It should be understood that the term “quadrupole configuration” as used herein does not require a perfect quadrupole but rather is intended to distinguish the film configuration from other configurations, such as a dipole configuration.

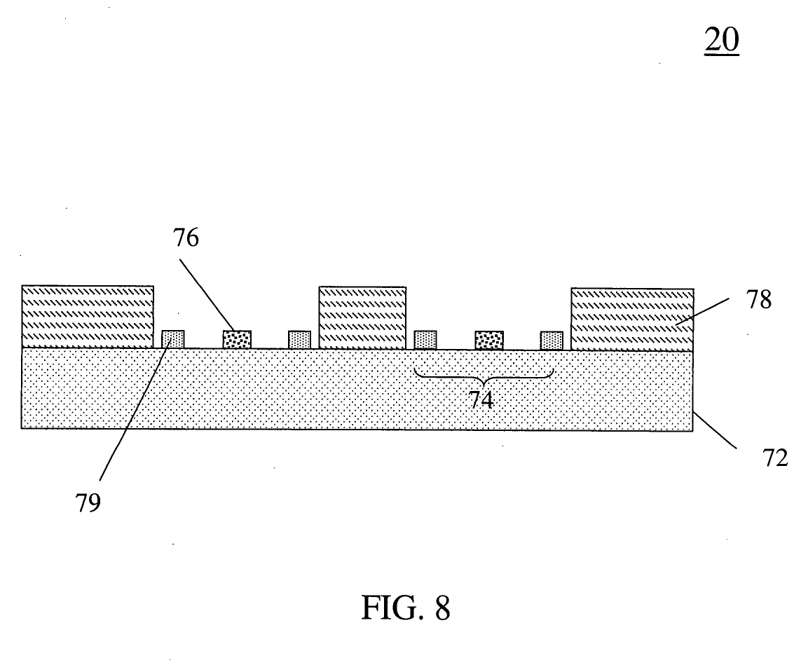

In order to provide electrical connections between receptor sites 74 for the respective functional blocks 10, for certain embodiments the article 20 further includes at least one interconnect layer 78 attached to the substrate 72, as schematically depicted in FIGS. 7 and 8, for example. FIG. 7 is a top view of an interconnect layer 78 and as shown, includes a number of connections 79 for interconnecting the functional blocks 10 to be assembled at the receptor sites 74. FIG. 8 is a side view of the article 20. Connections 79 can be formed of a variety of conductive materials, non-limiting examples of which include copper, gold and alloys thereof. Exemplary, non-limiting, interconnect layers include Copper on Kapton® and Gold on Kapton®.

Depending on the application, the receptor sites may be recessed within the substrate, may be level with the substrate 78 or may protrude from the substrate. In particular embodiments, one or more of the receptor sites are recessed, protrude and/or are level with the substrate. Further, the receptor sites 74 may be shaped. The receptor sites 74 may also be embossed within the substrate 72.

The substrate 72 may take many forms. For particular embodiments, the substrate 72 is flexible. In one non-limiting example, the flexible substrate 72 comprises polyimide. Other non-limiting examples include polycarbonate, liquid-crystal polymer and polyetherimide. Polyimide is an organic polymer, examples of which include materials marketed under the trade names Kapton® and Upilex®. Upilex® is commercially available from UBE Industries, Ltd., and Kapton® is commercially available from E. I. du Pont de Nemours and Company. According to a particular embodiment, the substrate comprises a sheet of a flexible material, such as polyimide. Such flexible substrates desirably lend themselves to low-cost manufacture of the assembly 20 using roll-to-roll fabrication techniques. Roll-to-roll fabrication techniques employ a variety of processes, non-limiting examples of which include gravure printing, flexo printing, ink jet printing, screen printing and offset printing. Other roll-to-roll fabrication processes utilize processes adapted from traditional batch processes such as photolithography, sputtering and wet chemical etching. Other benefits to the use of flexible substrates 72 include providing a robust article 20, as compared to conventional articles formed on rigid silicon or glass substrates, for example.

For other applications, the substrate 72 may be rigid, non-limiting examples of which include silicon and glass. In addition to being applicable to a wide variety of substrate materials, the substrate may have a variety of geometries and shapes. For example, for certain embodiments, the substrate 72 is a curved, rigid object, non-limiting examples of which include, for example, turbine blades and aircraft fuselages.

The coercivity H_{c} of the patterned magnetic film 76 should be selected such that the magnetic films 74 preserve their magnetic moments μ during assembly of the functional blocks 10 to the article 20. As used here, the coercivity H_{c} is the applied magnetic field required to reverse the magnetic moment μ. For particular embodiments, the patterned magnetic film 76 is characterized by a coercivity H_{c} of at least about ten Oersteads (Oe). According to more particular embodiments, the coercivity (H_{c}) is at least about thirty Oe, and still more particularly, at least about one hundred (100) Oe, and still more particularly, at least about one thousand (1000) Oe.

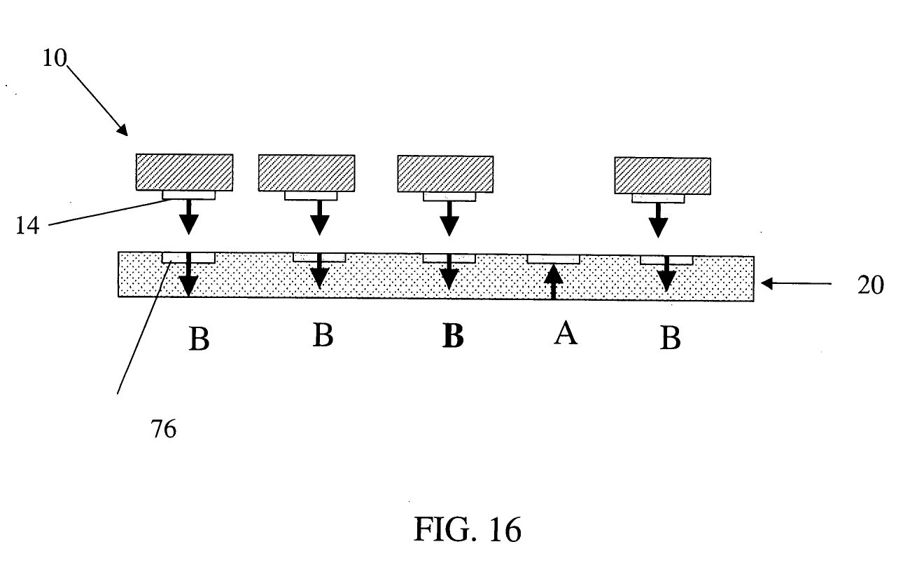

In order to self-assemble a wide-variety of electronic and other devices and structures, a heterogeneous self-assembly process is needed. However, heterogeneous self-assembly (namely, the self-assembly of building blocks with different types of elements) presents challenges that other self-assembly techniques have been unable to satisfactorily resolve. Beneficially, the present invention overcomes these challenges and may be used to assemble blocks having different types of elements to desired locations on the article 20. FIG. 16 illustrates one embodiment of an article 20 configured for heterogeneous self-assembly. As indicated, for example, in FIG. 16, the article 20 includes a number of receptor sites 74, and the patterned magnetic film 76 comprises a number of magnetic regions 76. A subset of receptor sites 74 (designated “B” in FIG. 16) have magnetic regions 76 configured in a binding configuration, and another subset of receptor sites 74 (designated “A” in FIG. 16) have magnetic regions 76 configured in an anti-binding configuration. Each of the subsets A, B includes at least one receptor site 74. As indicated, for example, in FIG. 16, the sites with regions 76 configured in a binding configuration will bind a functional block 10, whereas the sites 74 with regions 76 configured in an anti-binding configuration will not. In this manner, functional blocks 10 with certain elements (or groups of elements) may be positioned at specific receptor sites on the article, as desired. As described in copending, U.S. Patent Application to W. H. Huber et al., entitled “Methods for magnetically directed self assembly,” the magnetic regions 76 on the article can then be reconfigured to activate a new subset of binding sites for assembly of another set of functional blocks with certain elements (or groups of elements) to the new binding sites. This process can be repeated for each of the different types of functional blocks (that is, blocks with different types of elements). In this manner, the article 20 can be used to self-assemble a variety of elements 12 in a desired configuration, for example to self-assemble an electronic circuit or x-ray panel. As indicated, for example in FIG. 16, for a dipole configuration, a binding configuration comprises parallel magnetic moments, and an anti-binding configuration comprises anti-parallel magnetic moments. For one exemplary quadrupole configuration, a binding configuration would correspond to a net quadrupole moment, and an anti-binding configuration would correspond to the absence of a net quadrupole moment.

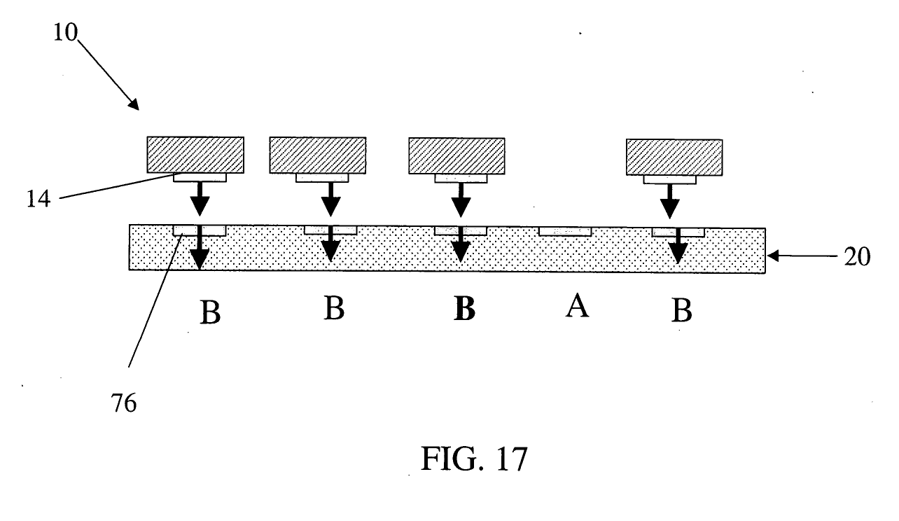

FIG. 17 illustrates another embodiment of an article 20 configured for heterogeneous self-assembly. As indicated, for example, in FIG. 17, the article 20 includes a number of receptor sites 74, and the patterned magnetic film 76 comprises a number of magnetic regions 76. A subset of the receptor sites 74 (also labeled “B”) have magnetic regions 76 configured in a binding configuration, and another subset of receptor sites 74 (“A”) have magnetic regions 76 with no remnant magnetization. Each of the subsets comprises at least one receptor site 74. As used herein, the phrase “no remnant magnetization” means a remnant magnetization less than approximately half the remnant magnetization of the ‘B’ binding sites. As indicated, for example, in FIG. 17, the sites with regions 76 configured in a binding configuration (“B”) will bind a functional block 10, whereas the sites 74 with regions 76 without remnant magnetization (“A”) will not. In this manner, functional blocks 10 with certain elements (or groups of elements) may be positioned at specific receptor sites on the article, as desired. One technique to form this article embodiment would be to configure all of the sites at zero remnant magnetization and then selectively activate a subset of the sites, as described in copending, U.S. Patent Application to W. H. Huber et al., entitled “Methods for magnetically directed self assembly.” After self-assembly of a first set of functional blocks to the article 20, the magnetic regions 76 on the article can then be reconfigured to activate a new subset of binding sites for assembly of another set of functional blocks with certain elements (or groups of elements) to the new binding sites. This process can be repeated for each of the different types of functional blocks (that is, blocks with different types of elements). In this manner, the article 20 can be used to self-assemble a variety of elements 12 in a desired configuration, for example to self-assemble an electronic circuit, x-ray panel or display.

An assembly embodiment of the invention is described generally with reference to FIGS. 1, 2, 9. As shown for example, in FIGS. 1 and 2, assembly 30 includes at least one functional block 10. As indicated, for example, in FIG. 2, the functional block 10 includes at least one element 12 and a patterned magnetic film 14 comprising at least one magnetic region 14. The assembly 30 further includes an article 20 comprising a substrate 72, at least one receptor site 74 disposed on the substrate 72 and at least one receptor 76 configured to generate a magnetic field gradient for attracting the magnetic region. At least one receptor 76 is positioned at the receptor site 74.

FIG. 2 shows a partially assembled assembly 30. For the exemplary embodiment shown in FIG. 2, the assembly 30 further includes insulating regions 86 disposed on the substrate 72 between neighboring functional blocks 10.

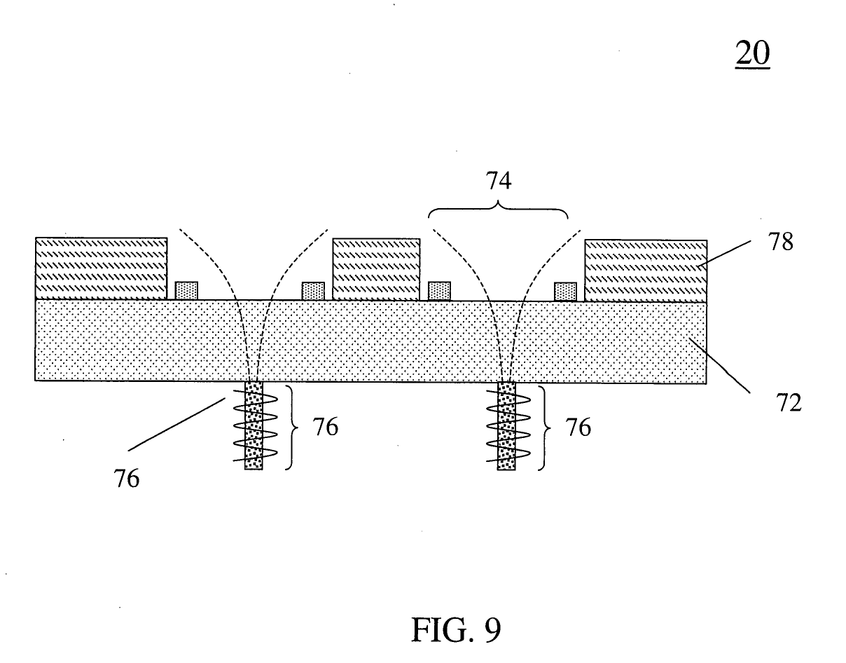

Various embodiments of the article 20 are described above. For the particular embodiments illustrated in FIGS. 1 and 2, for example, the receptor 76 comprises a patterned magnetic film 76 comprising a material with a perpendicular magnetic anisotropy. According to a more particular embodiment, the patterned magnetic film 76 comprises a multilayer structure. In order for the magnetic films 76 to preserve their magnetic moments μ during assembly, the patterned magnetic film 76 is characterized by a coercivity H_{c} of at least about ten Oersteads Oe, for particular embodiments. According to a more particular embodiment, the coercivity H_{c} is at least about one hundred Oe. For the exemplary embodiment depicted in FIG. 9, for example, the receptor 76 comprises an electromagnet, for example, a coil or a coil plus a permanent magnet.

The functional blocks 10 are described in detail in concurrently filed U.S. Patent Application to Huber et al., “Functional blocks for assembly and method of manufacture.” In addition, various aspects of the functional blocks 10 are discussed below with reference to FIGS. 2 and 10-15.

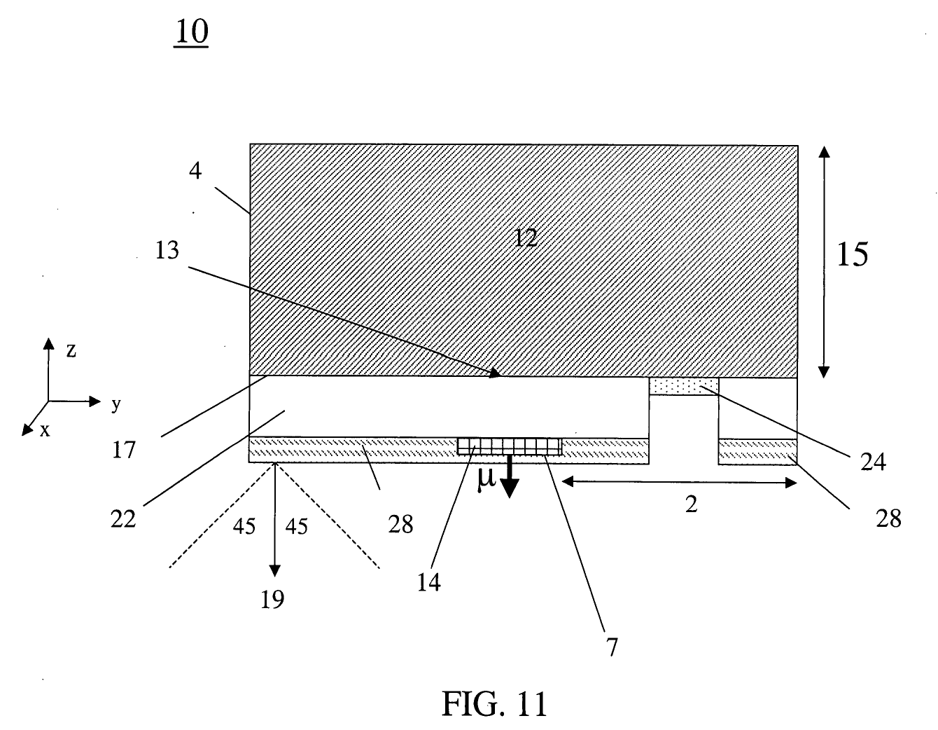

The patterned magnetic film 14 for the various functional blocks 10 may comprise a single or multiple magnetic regions 14. For example, for the exemplary embodiment shown in FIG. 11, the patterned magnetic film 14 for the functional block 10 comprises a single magnetic region 14. In contrast, for the exemplary embodiment shown in FIG. 2, for example, the patterned magnetic film 14 for the functional block 10 comprises multiple magnetic regions 14. Different functional blocks 10 may have different configurations for the patterned magnetic film 14.

According to a particular embodiment, the patterned magnetic film 14 comprises a material with a perpendicular magnetic anisotropy. For more particular embodiments, the patterned magnetic film 14 comprises a multilayer structure, as illustrated by FIG. 10, for example. As discussed in Huber et al., “Functional blocks for assembly and method of manufacture,” the coercivity H_{c} of the patterned magnetic film 14 should be selected such that the magnetic films 14 preserve their magnetic moments μ for assembly to the article 20. As used here, the coercivity H_{c} is the applied field required to reverse the magnetic moment μ. According to a particular embodiment, the patterned magnetic film 14 is characterized by a coercivity (H_{c}) of at least about ten Oersteads (Oe). According to more particular embodiments, the coercivity (H_{c}) is at least about thirty Oe, more particularly at least about one hundred Oe, and still more particularly, at least about one thousand (1000) Oe.

As discussed in Huber et al., “Functional blocks for assembly and method of manufacture,” the element 12 has a connecting surface 17, and the magnetic region 14 is configured to exhibit a magnetic moment μ that is oriented in a range of plus or minus forty-five degrees from a surface normal 19 for the connecting surface 17. By “configured” it is meant that the specified magnetic moment μ can be generated by application of a suitably large magnetic field and after such generation remains even in the absence of an applied magnetic field. As indicated, for example in FIG. 1, the connecting surface 17 is the surface of the element 12 configured to face the article 20. According to a more particular embodiment, the magnetic moment μ is oriented in a range of plus or minus twenty degrees, and more particularly, in a range of plus or minus ten degrees, from the surface normal 19 for the connecting surface 17. For the exemplary embodiment of FIG. 1, the magnetic moment μ is aligned along the surface normal 19 for the connecting surface 17. Beneficially, by aligning the magnetic moment μ of the patterned magnetic film 14 along the surface normal 19, connecting surfaces 17 of multiple elements 12 will repulse each other. In this manner, the tendency of the functional blocks 10 to agglomerate when dispensed in slurry, is reduced.

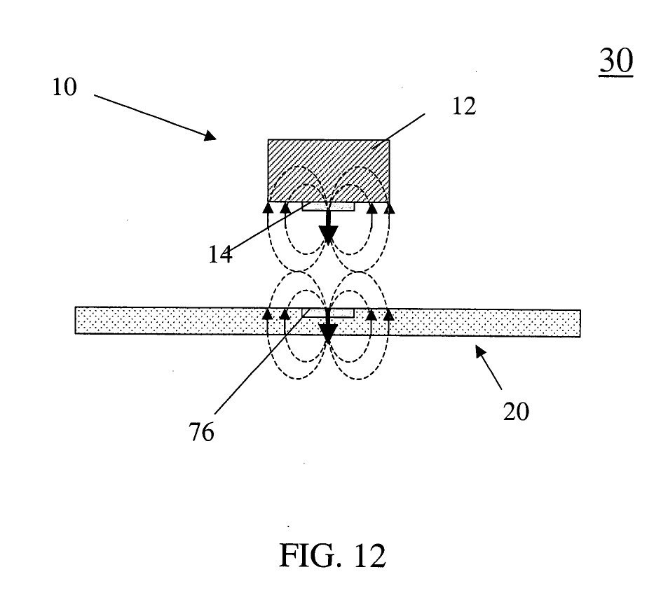

FIG. 12 illustrates an exemplary dipole binding configuration for the magnetic moment μ of the patterned film 14. For the exemplary embodiment depicted in FIG. 12, the patterned magnetic film 14 is arranged in a dipole configuration and comprises a material with a perpendicular magnetic anisotropy. It should be understood that the term “dipole configuration” as used herein does not require a perfect dipole but rather is intended to distinguish the film configuration from other configurations, such as a quadrupole configuration.

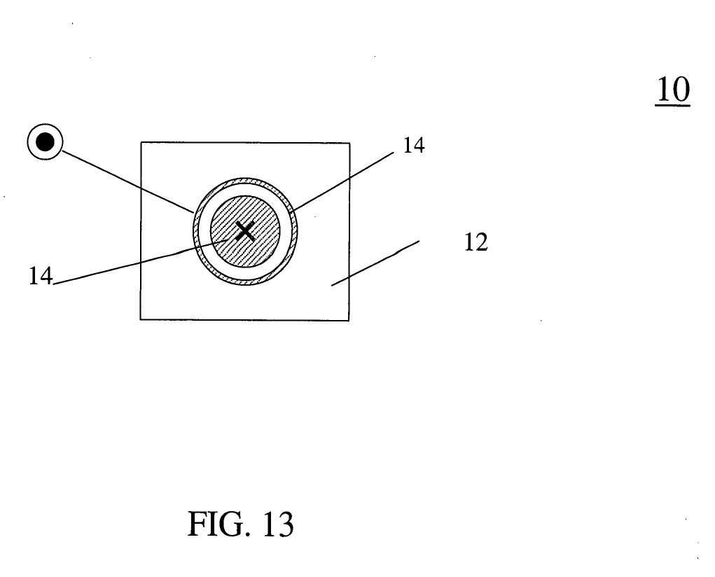

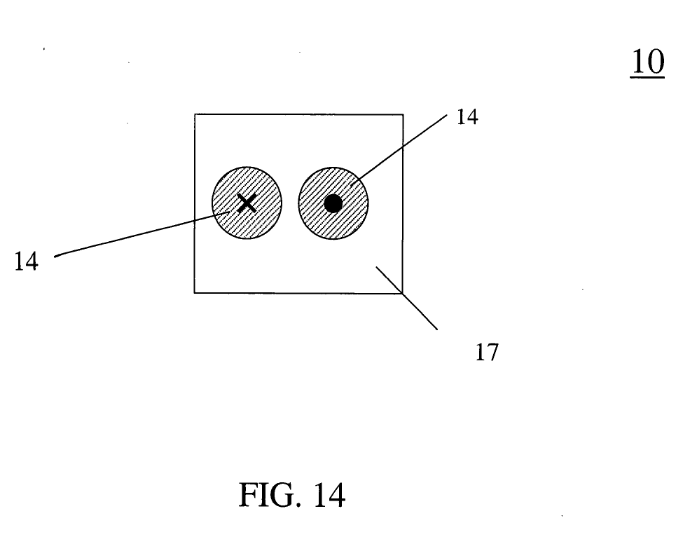

FIGS. 13 and 14 illustrate two exemplary quadrupole configurations for the magnetic moment μ of the patterned magnetic film 14. FIGS. 13 and 14 show the connecting surface 17 of the element 12 with the magnetic regions 14 arranged thereon. For the exemplary embodiment depicted in FIGS. 13 and 14, the patterned magnetic film 14 comprises a number of magnetic regions 14 arranged in a quadrupole configuration. For the exemplary configuration of FIG. 13, the inner and outer magnetic regions 14 have the same area (and hence volume) and opposite directions of magnetization. Similarly, for the exemplary configuration of FIG. 14, the neighboring regions 14 have the same area (and hence volume) and opposite direction of magnetization. According to a more particular embodiment, the patterned magnetic film 14 comprises a material with a perpendicular magnetic anisotropy.

As discussed in Huber et al., “Functional blocks for assembly and method of manufacture,” in order to prevent agglomeration of the functional blocks 10 in the slurry during assembly, “pull-back” of the magnetic regions 14 is desirable. For the exemplary embodiment of FIG. 11, for example, the patterned magnetic film 14 is disposed in an interior 13 of the element 12. As used here, the term “interior” is used relative to the X-Y plane and is intended to be distinguished from the perimeter of the element. According to a particular embodiment, a gap 2 between the magnetic region 14 and a perimeter 4 of the element 12 is at least about 0.1 tₑ, where tₑ is the thickness 15 of the element 12, as noted above. As used here, the gap 2, is the minimum value of the distance between the region 14 and the perimeter 4. For example, for a circular region 14 and a rectangular perimeter 4, the distance between the region and the perimeter varies. The gap 2 is defined to be the minimum value of the distance between the region 14 and the perimeter 4. According to a more particular embodiment, the gap 2 is at least about 0.25 tₑ, and still more particularly, the gap is at least about 0.4 tₑ. The desired pull-back is determined at least in part by the magnitude of the magnetic moment μ of the magnetic regions 14. Although pull-back is generally desirable, for other configurations (not shown), the regions 14 may be disposed along the perimeter of the element 12.

The present invention can be used with a wide variety of elements 12, and exemplary elements 12 include without limitation semiconductor devices, passive elements, photonic band-gap elements, luminescent materials, sensors, micro-electrical mechanical systems (MEMS) and energy harvesting devices (such as photovoltaic cells). As used here, the term “passive element” should be understood to refer to passive circuit elements, non-limiting examples of which include resistors, capacitors, inductors, and diodes. Exemplary semiconductor devices 12 include, without limitation, transistors, diodes, logic gates, amplifiers and memory circuits. Examples of transistors include, without limitation, field effect transistors (FETs), MOSFETs, MISFETs, IGBTs, bipolar transistors and J-FETs. The semiconductor devices may for example comprise Si, GaN, GaAs, InP, SiC, SiGe or other semiconductors.

A functional block 10 may include a single element 12 or a group of elements 12. A group of elements 12 for a functional block 10 may include different types of elements. For example, a functional block may comprise multiple transistors configured as a digital logic gate or an analog amplifier.

In one example, the element 12 includes a field effect transistor formed on single-crystal silicon (not shown). Efforts are on-going to create high-quality, large-area devices, such as displays and x-ray detectors. However, the formation of a large-area array of high-quality transistors is a limiting factor. Silicon wafer processing currently produces the highest quality transistors, but the wafers are limited in size (typically about 300 mm). Larger arrays of transistors can be formed using amorphous-silicon (α-Si) or poly-silicon (poly-Si). However, α-Si and poly-Si transistors are typically characterized by low mobility, large feature size (the channel length, which is the distance between the source and the drain, is typically on the order of 4 microns for commercial devices) and the gate dielectric is typically of relatively low quality. Consequently, the α-Si and poly-Si transistors typically are slow, exhibit poor gain and drift, relative to single crystal Si transistors. Further, both the substrates used to fabricate α-Si and poly-Si based thin film transistors are also limited in size, such that arrays produced using these techniques are typically less than about 2 meters by 2 meters in area. Beneficially, the functional blocks 10 can be used to assemble to an article 20 to provide the performance benefits of single-crystal silicon FETs at a low cost for the larger article 20. Because the FETs are formed in a separate process and assembled to the article 20, there is no upper limit on the size of the article 20. Further, because the cost per unit area of assembled substrate is dictated by the density of functional blocks 10 and the cost of the article 20, by utilizing small-area functional blocks 10, high quality FETs can be assembled to large area articles (for example 10 m×10 m) at relatively low cost.

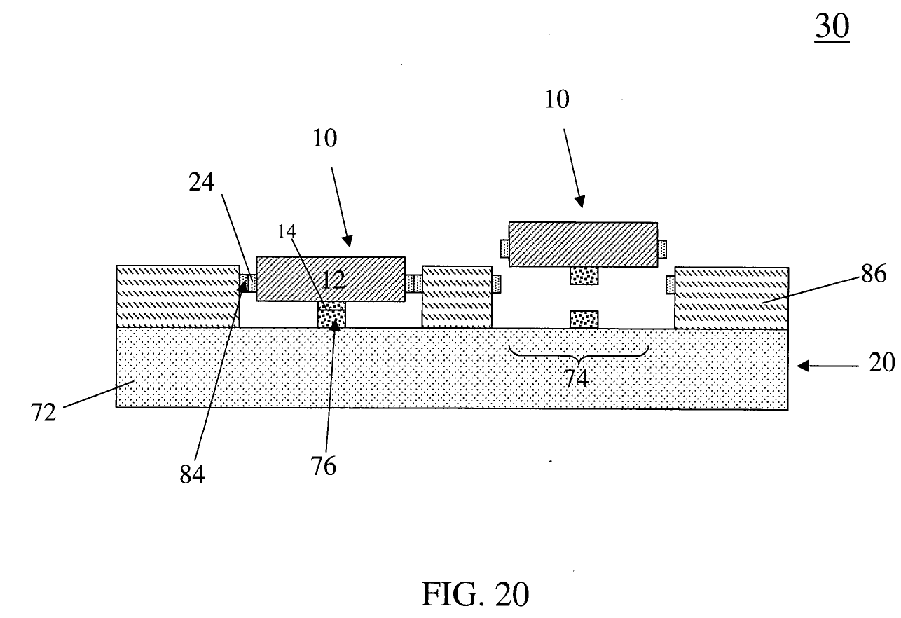

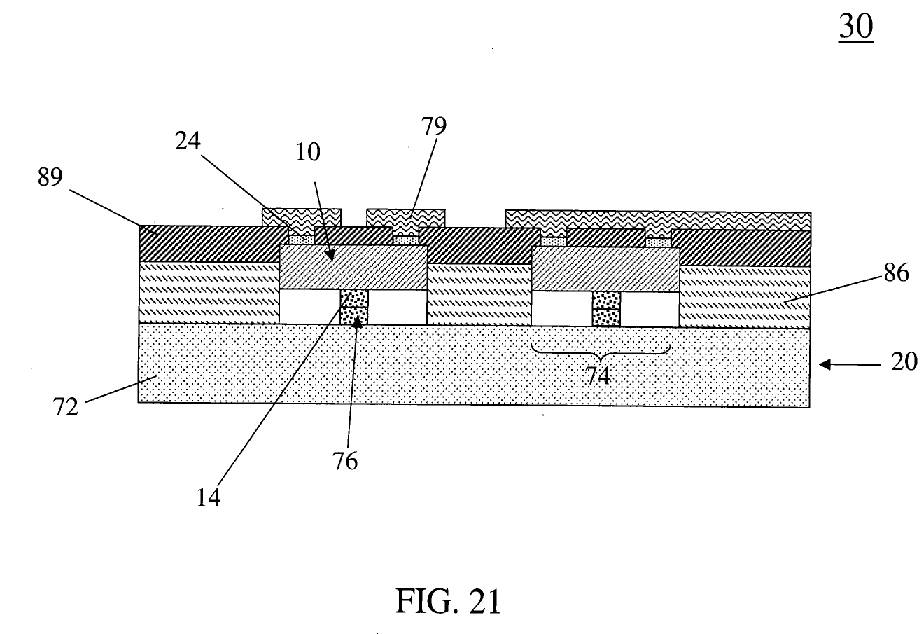

Many of these elements 12, such as the semiconductor devices, require electrical contacts. For many embodiments, the assembly 30 further includes at least one contact 24, 84, for the element(s) 12. For the exemplary embodiment depicted in FIG. 11, for example, the contact(s) 24 are formed on the functional block 10. For the exemplary embodiments shown in FIGS. 2, 20 and 21 for example, contacts 24, 84 are formed on the functional blocks 10 and on the article 20. For the example embodiment shown in FIG. 2, the contacts 24, 84 are front contacts. For the example embodiment shown in FIG. 20, the contacts 24, 84 are side contacts. For the example embodiment shown in FIG. 21, the contacts 24, 84 are back contacts, and the assembly 30 further includes planarizing layer 89 and connections 79. In other embodiments (not expressly shown), the configuration for contacts 24, 84 combines front, and/or side and/or back contact configurations.

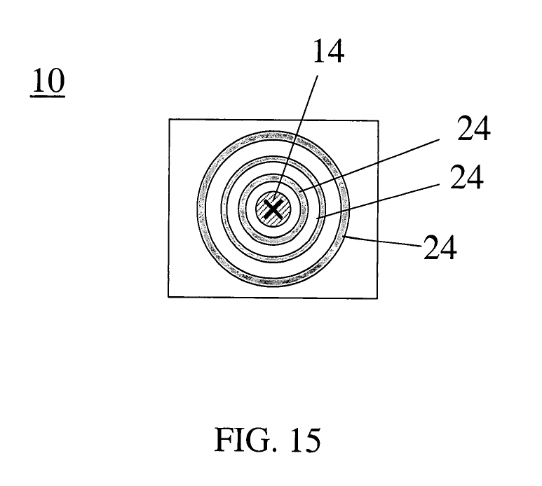

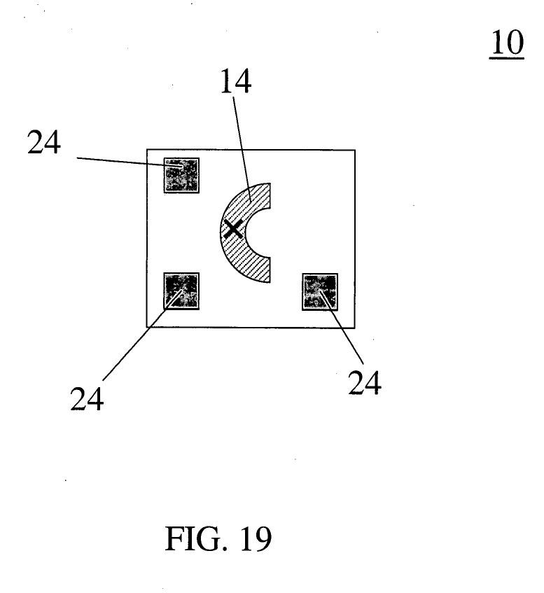

The contacts 24, 84 are formed of conductive materials, non-limiting examples of which include gold, platinum, nickel, copper, aluminum, titanium, tungsten, tantalum, molybdenum and alloys. The contacts 24, 84 can be configured as desired. For example, for the exemplary embodiment shown in FIG. 15, the magnetic region 14 is circular, and the contacts 24 are formed as rings centered on the magnetic region. By using contacts with circular symmetry, magnetic regions can be utilized with circular symmetry. Alternatively, if the magnetic regions 14 are not symmetric, as shown for example in FIG. 19, the contacts 24 do not require circular symmetry.

After assembly, it is desirable to fasten the functional block 10 to the article 20, for example by solder or other fastening means. According to a particular embodiment, the at least one contact 24, 84 is configured to fasten the functional block to an article 20 after assembly of the functional block to the article 20. Non-limiting examples of contact materials further include solders, such as Indium, Tin, Lead, Bismuth, Silver, Cadmium, Zinc and various alloys. The solder may be deposited on a Gold or other conductive film, for example, forming a layered structure. The solder may be deposited on the contacts 24 to the functional blocks 10 and/or deposited on the article 20.

As shown, for example, in FIG. 11, for particular embodiments, the functional block 10 further includes a protective layer 22 configured to protect the functional block 12. For the exemplary configuration shown in FIG. 11, the protective layer 22 is formed over portions of element 12. The protective layer 22 can be organic or inorganic, and example materials for the protective layer 22 include, without limitation, Si₃N₄ (silicon nitride), SiO₂ (silicon dioxide), polyimide, BCP and paraylene. Polyimide is an organic polymer, examples of which include materials marketed under the trade names Kapton® and Upilex®. Upilex® is commercially available from UBE Industries, Ltd., and Kapton® is commercially available from E. I. du Pont de Nemours and Company. Other exemplary flexible organic polymers include polyethersulfone (PES) from BASF, polyethyleneterephthalate (PET or polyester) from E. I. du Pont de Nemours and Company, polyethylenenaphthalate (PEN) from E. I. du Pont de Nemours and Company, and polyetherimide (PEI) from General Electric.

As discussed above, solder is used for certain embodiments to fasten the functional blocks 10 to the article 20 after assembly. For other embodiments, and as shown, for example, in FIG. 11, the assembly 30 further includes an activated adhesive 28 that fastens the functional block(s) 10 to the article 20. For the exemplary embodiment depicted in FIG. 11, for example, the functional block 10 further includes an activated adhesive 28 attached (indirectly) to the element 12 and configured to fasten the functional block 10 to an article 20 after assembly of the functional block to the article and upon activation. The adhesive 28 may be attached directly or indirectly to the element 12, and, for the example shown in FIG. 11, is attached indirectly to the element 12. In other embodiments (not shown), the article 20 includes an activated adhesive configured to fasten the article 20 to the functional blocks 10 after assembly of the functional blocks to the article and upon activation. Examples of activated adhesives 28 include, without limitation, photopolymerizable acrylate adhesives. Depending on the adhesive used, the activation may comprise application of ultraviolet light or thermal activation, for example. Other adhesives may be chemically activated.

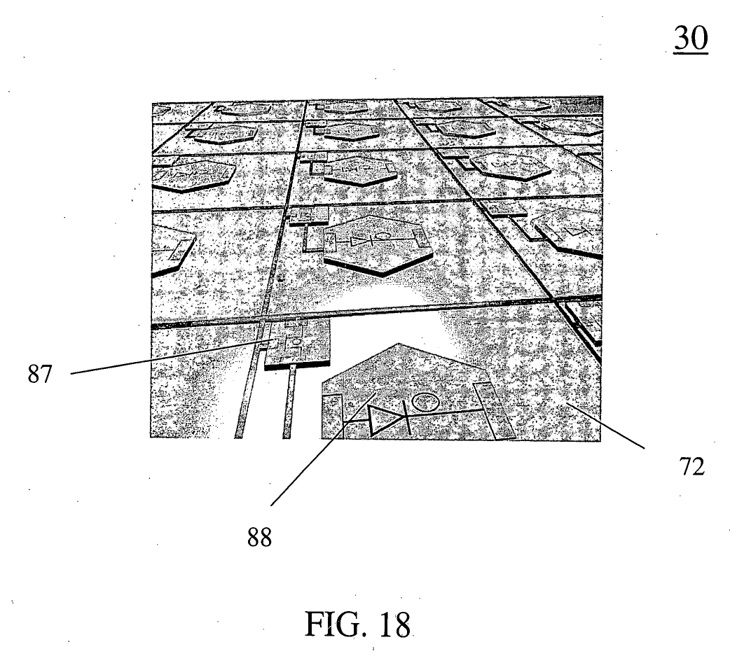

A heterogeneous assembly 30 embodiment is described with reference to FIG. 18. As indicated in FIG. 18, for example, the heterogeneous assembly 30 includes a number of receptor sites 74 disposed on the substrate 72, a number of receptors 76 disposed within respective ones of the receptor sites 74, and a number of functional blocks 10 corresponding to N block types disposed at respective ones of the receptor sites 72. N is an integer and N≧2. For the exemplary embodiment shown in FIG. 18, N=2, the first type of element is a transistor 87, and the second type of element is a diode 88. For one exemplary display embodiment, the diode 88 is a light emission diode (LED), and the substrate 72 may be rigid (for example, silicon or glass) or may be flexible (for example, a polymer sheet). For one exemplary x-ray panel embodiment, the diode 88 is a photodiode, and the substrate may be rigid (for example silicon or glass) or may be flexible (for example a polymer sheet).

Although only certain features of the invention have been illustrated and described herein, many modifications and changes will occur to those skilled in the art. It is, therefore, to be understood that the appended claims are intended to cover all such modifications and changes as fall within the true spirit of the invention.

1. An article for assembly comprising:
a substrate;
at least one receptor site disposed on said substrate; and
a patterned magnetic film comprising at least one magnetic region, each magnetic region being disposed within one of said receptor sites, said patterned magnetic film comprising a material with a perpendicular magnetic anisotropy.

2. The article of claim 1, wherein each magnetic region is configured to generate a magnetic field gradient.

3. The article of claim 2, wherein the magnetic field gradient is configured to attract a patterned magnetic film for a block having a magnetic moment, oriented in a range of plus or minus forty-five degrees from a surface normal for the patterned magnetic film for the block.

4. The article of claim 1, comprising a plurality of receptor sites, wherein said patterned magnetic film comprises a plurality of magnetic regions, wherein at least one of said magnetic regions is disposed at each of said receptor sites.

5. The article of claim 1, comprising a plurality of receptor sites, wherein said patterned magnetic film comprises a plurality of magnetic regions, wherein a plurality of said magnetic regions are arranged in a quadrupole configuration for at least one of said receptor sites.

6. The article of claim 1, further comprising at least one interconnect layer attached to said substrate.

7. The article of claim 1, wherein at least one receptor site is recessed within said substrate.

8. The article of claim 1, wherein at least one receptor site protrudes from said substrate.

9. The article of claim 1, wherein said substrate is flexible.

10. The article of claim 1, wherein said substrate is a curved, rigid object.

11. The article of claim 1, wherein said patterned magnetic film is characterized by a coercivity (H_{c}) of at least about ten Oersteads (Oe).

12. The article of claim 11, wherein the coercivity (H_{c}) is at least about one hundred Oe.

13. The article of claim 12, wherein the coercivity (H_{c}) is at least about one thousand (1000) Oe.

14. The article of claim 1, comprising a plurality of receptor sites, wherein said patterned magnetic film comprises a plurality of magnetic regions, wherein a subset of said receptor sites have magnetic regions configured in a binding configuration, wherein another subset of said receptor sites have magnetic regions configured in an anti-binding configuration, and wherein each of the subsets comprises at least one receptor site.

15. The article of claim 1, comprising a plurality of receptor sites, wherein said patterned magnetic film comprises a plurality of magnetic regions, wherein a subset of said receptor sites have magnetic regions configured in a binding configuration, wherein another subset of said receptor sites have magnetic regions with no remnant magnetization, and wherein each of the subsets comprises at least one receptor site.

16. An assembly comprising:
at least one functional block comprising at least one element and a patterned magnetic film comprising at least one region; and
an article comprising a substrate, at least one receptor site disposed on said substrate and at least one receptor configured to generate a magnetic field gradient for attracting said region, said at least one receptor being positioned at said receptor site.

17. The assembly of claim 16, wherein said patterned magnetic film comprises a material with a perpendicular magnetic anisotropy.

18. The assembly of claim 17, wherein said patterned magnetic film for at least one of said at least one functional blocks comprises a multilayer structure.

19. The assembly of claim 18, wherein said patterned magnetic film is characterized by a coercivity (H_{c}) of at least about ten Oersteads (Oe).

20. The assembly of claim 19, wherein the coercivity (H_{c}) is at least about one hundred Oe.

21. The assembly of claim 20, wherein the coercivity (H_{c}) is at least about one thousand (1000) Oe.

22. The assembly of claim 16, wherein said element has a connecting surface, and wherein said patterned magnetic film is configured to exhibit a magnetic moment (μ) that is oriented in a range of plus or minus forty-five degrees from a surface normal for said connecting surface.

23. The assembly of claim 22, wherein the magnetic moment (μ) is oriented in a range of plus or minus twenty degrees from the surface normal for said connecting surface.

24. The assembly of claim 23, wherein the magnetic moment (μ) is oriented in a range of plus or minus ten degrees from the surface normal for said connecting surface.

25. The assembly of claim 16, wherein said patterned magnetic film is arranged in a dipole configuration and comprises a material with a perpendicular magnetic anisotropy.

26. The assembly of claim 16, wherein said patterned magnetic film for at least one of said at least one functional blocks comprises a plurality of regions.

27. The assembly of claim 16, wherein said patterned magnetic film is disposed in an interior of said element.

28. The assembly of claim 27, wherein said element is characterized by a thickness tₑ, and wherein a gap between said magnetic region and a perimeter of said element is at least about 0.25 tₑ.

29. The assembly-of claim 16, further comprising at least one contact for said element.

30. The assembly of claim 29, wherein said patterned magnetic film is circular, and wherein said at least one contact is formed as a ring centered on said patterned magnetic film.

31. The assembly of claim 29, wherein said at least one contact fastens said functional block to said article.

32. The assembly of claim 16, wherein said at least one functional block further comprises a protective layer configured to protect said functional block.

33. The assembly of claim 16, further comprising an activated adhesive that fastens said functional block to said article.

34. The assembly of claim 16, wherein said at least one element is selected from the group consisting of a semiconductor device, a passive element, a photonic bandgap element, a luminescent material, a sensor, a micro-electrical mechanical system (MEMS), an energy harvesting device and combinations thereof.

35. The assembly of claim 34, wherein said at least one element comprises a semiconductor device selected from the group consisting of transistors, diodes, logic gates, amplifiers and memory circuits.

36. The assembly of claim 34, wherein said at least one element comprises a passive element selected from the group consisting of resistors, capacitors, inductors, and diodes.

37. The assembly of claim 16, wherein said patterned magnetic film comprises a plurality of regions arranged in a quadrupole configuration.

38. The assembly of claim 16 comprising a plurality of receptor sites disposed on said substrate, a plurality of receptors disposed within respective ones of said receptor sites, and a plurality of functional blocks corresponding to N block types disposed at respective ones of said receptor sites, wherein N is an integer and N≧2.

39. The assembly of claim 38, wherein a first one of the block types comprises transistor blocks, wherein a second one of the block types comprises photodiode blocks, and wherein the transistor blocks and photodiode blocks are arranged in an x-ray panel configuration.

40. The assembly of claim 39, wherein the substrate comprises a rigid substrate.

41. The assembly of claim 39, wherein said substrate comprises a flexible substrate.

42. The assembly of claim 38, wherein a first one of the block types comprises transistor blocks, wherein a second one of the block types comprises light emitting diode blocks, and wherein the transistor blocks and light emitting diode blocks are arranged in a display configuration.

43. The assembly of claim 42, wherein the substrate comprises a rigid substrate.

44. The assembly of claim 42, wherein said substrate comprises a flexible substrate.

45. The assembly of claim 16, wherein said at least one receptor comprises a patterned magnetic film comprising a material with a perpendicular magnetic anisotropy.

46. The assembly of claim 45, wherein said patterned magnetic film for said receptor site comprises a multilayer structure.

47. The assembly of claim 45, wherein said patterned magnetic film for said receptor site is characterized by a coercivity (H_{c}) of at least about ten Oersteads (Oe).

48. The assembly of claim 47, wherein the coercivity (H_{c}) is at least about one hundred Oe.

49. The assembly of claim 16, wherein said at least one receptor comprises an electromagnet.
